(19) United States
(12) Patent Application Publication
(10) Pub. No.: US 2023/0102533 A1
(43) Pub. Date: Mar. 30, 2023

(54) STARCH PROPIONATE USED AS A FOOD ADDITIVE AND/OR DIETARY SUPPLEMENT FOR PREVENTION OF OVERWEIGHT AND OBESITY

(71) Applicants: Ole Abilgaard, Hosterkob (DK); Theis Wilbek, Kobenhavn Nv (DK)

(72) Inventors: Ole Abilgaard, Hosterkob (DK); Theis Wilbek, Kobenhavn Nv (DK)

(21) Appl. No.: 17/801,199

(22) PCT Filed: Feb. 13, 2021
(86) PCT No.: PCT/DK2021/050041

§ 371 Date: Aug. 19, 2022

(30) Foreign Application Priority Data
Feb. 21, 2020 (DK) PA 2020 00215

(51) Int. Cl. A61K 31/19; A23L 33/12; A61K 47/26

(52) U.S. Cl. CPC A61K 31/19; A23L 33/12; A61K 47/26

(57) ABSTRACT

Starch propionate for humans is provided for weight loss and prevention of overweight and obesity. Starch propionate functions as a platform for delivery of propionic acid in the colon. It passes un-degraded to the colon, where propionic acid is released. Propionic acid in the colon stimulates the intestinal membrane to release appetite suppressing hormones GLP-1,(GLP-2) and PPY that reduces calorie intake and inhibits the production of fat in the liver and fat-tissue. Increased propionic acid in the blood inhibits fat production in the liver and fat-tissue. It also promotes the development of the gastrointestinal tract. Starch propionate can be used to reduce the appetite and calorie intake in humans.

### TECHNICAL FIELD

The invention concerns the application of starch propionate as food additive and/or dietary supplement for prevention of overweight and obesity. Starch propionate used as a food additive and/or dietary supplement can be used to weight reduction, prevention of overweight and obesity, decrease the appetite, reduce the food- and calorie intake, lower the intra-abdominal fat distribution, for lowering of the amount of intrahepatocellular fat and for reduction of the desire for high energy foods in humans. Starch propionate is starch esterified with propionic acid in varying degrees. Starch consists of two different polymers, amylose and amylopectin, and can originate from different sources—e.g. from potatoes, rice, wheat and maize. Depending on the source, the ratio of amylose and amylopectin can vary. Amylose is a linear polymer of glucose units joined by α-1,4-glucosidic bonds with varying length. Amylopectin is likewise a polymer of glucose units joined by α-1,4-glucosidic bonds, but additionally also has branches to other α-1,4-glucosidic chains by α-1,6-glucosidic bonds. The degree of branching is often in the magnitude of 5%, but it can vary.

### BACKGROUND ART

Propionic acid is one of the three ‘short-chain fatty acids’ (SCFAs) that are natural sources of energy for humans and animals. The three SCFAs are acetic acid, propionic acid and butyric acid and are known to have health promoting effects in humans and animals. SCFAs are primarily produced by fermentation of dietary fibers by bacteria in the colon. Dietary fibers are not degraded by enzymes in the small intestine and thus are available for fermentation by the bacteria present in the colon. There are several types of dietary fibers but not all can be fermented by bacteria in the colon. When dietary fibers are fermented, SCFAs are produced and absorbed by the intestine and in this way SCFAs are included in the metabolism as a natural energy source. SCFAs additionally activates different types of receptors found on the cells comprising the intestinal membrane and intestinal wall. One of the consequences of this is that peptide hormones, like GLP1, GLP2 and PPY, are released from L cells found in the intestinal epithelium/intestinal membrane. Additionally the general health of the intestine is increased by the presence of SCFAs, by e.g. increasing the growth of the intestinal epithelium, tightening joints between the cells in the membrane and increasing the number of cells in the intestinal epithelium including the amount of L-cells. The peptide hormones GLP1 and PPY are known to affect the metabolism in humans and in animals; to lower and stabilize the blood sugar, increase insulin sensitivity and insulin production and increase satiety/decrease appetite. Circulating SCFAs in the blood stream also affect the metabolism in humans and animals, and can protect against overweight, inhibit the adipose tissue abilities to produce and accumulate fat, increase the metabolism and reduce the amount of fat in the blood.

Acetic acid, propionic acid and butyric acid participates differently in the metabolism. It is known that propionic acid promotes and participates in gluconeogenesis in the liver and the peripheral tissue, whereas acetic acid and butyric acid promote and participate in the formation of fat. Propionic acid and butyric acid have health promoting effects on the human metabolism, different to acetic acid in that propionic acid and butyric acid are more potent activators of the different SCFA-binding receptors found in the lower intestine (primarily in the colon). If acetic acid, propionic acid or butyric acid are added directly to the diet (ingested freely and orally), they will be absorbed rapidly in the upper part of the gastrointestinal tract and become metabolized. GLP1, GLP2 and PPY are primarily released from intestinal cells in the colon and thus SCFAs absorbed in the upper part of the gastrointestinal tract do not promote the release of GLP1, GLP2 and PPY from the intestinal cells in the colon. The effect caused by SCFAs of promoting the growth of the intestinal wall or membrane in the colon, will also only take place if SCFAs reach the colon, and thus promote the healthy tightening of the membrane and enlarging of the number of cells in the intestinal wall of the colon. Thus, the release of SCFAs in the colon is essential to achieve the positive effects described (release of GLP1 and PYY hormones, proliferation of L cells, tightening of the membrane etc.). This release of SCFAs directly in the colon is normally almost exclusively achieved by fermentation of dietary fibers by bacteria present in the colon and capable of producing these.

The overall amount and ratio of different SCFAs produced in the colon by fermentation depends on which bacteria are present and prolific. Modern western diet has a low content of dietary fibers, with the result that in humans in general on a western diet, too little production of SCFAs by fermentation in the colon takes place. The western diet also affects the composition of bacteria in the colon negatively, in a way that certain strains of bacteria specialized in fermenting fibers and producing SCFAs in some people are lacking all together—reducing the overall ability of their microbiome in the colon to produce SCFAs.

The composition of gut bacteria/microbiome in the colon is to a large extend locked or at least very difficult to change. Extensive research suggests that the composition of bacteria is formed in the first few years of life and is hereafter difficult to change by environmental impact, including: diet, medication, exercise as well as pro and antibiotic substances such as pro- and prebiotic dietary supplements and anti bacterial compounds found in hygiene products. For these and other reasons fermentation of dietary fibers and production of SCFAs in the colon of humans on a modern western diet, can be problematic and is a source of variation and uncertainty.

Effective fermentation in the colon of dietary fibers and ensuing production of SCFAs depends on the presence in particular of prevotella bacteria, which are widely found in natural populations like africans and south americans on a primary agrarian or plant-based diet. These bacteria are widely lacking in people eating a western diet. Research have found that children on a western diet in average only have 0-5% prevotella bacteria, while children on an african/south american plant-based diet on average have above 50% prevotella bacteria [1].

Recent research by professor Arne Astrup from Copenhagen University has shown that test persons on diets with a high amount of fiber including resistant starches (New Nordic Diet), only reduce weight if they have prevotella bacterias present in the colon. Additionally the same investigations showed that the bacterial composition was fairly constant, so that test persons lacking prevotella bacterias, did not gain these in an effective or meaningful measure during the study as a result of the altered diet rich in fibers [2].

In patent 2013-07-31PCT/GB2013/052056 it is described how the consumption of propionylated inulin in humans reduce appetite and reduce food and calorie intake [3][4]. Inulin, a natural dietary fiber, is fermented in the colon in humans and is a polysaccharide that primarily consists of fructose units. Propionylated inulin is inulin esterified with propionic acid. When eaten it also is not degraded in the small intestine, but reaches the colon undigested. In the colon the propionic esters are cleaved into inulin and propionic acid. The propionic acid is absorbed in the colon and the free inulin can afterwards be fermented producing additional SCFAs in the colon. The oral ingestion of propionylated inulin in a clinical trial, for an extended period has shown (NCT00750438) that propionylated inulin prevents weight gain, reduces food and calorie intake, reduces the intra-abdominal fat-distribution and reduces the amount of intrahepatocellular fat. Additionally, it was shown that ingestion of propionylated inulin reduced the desire for high-energy foods by affecting the central nervous system in the tested humans [5].

Data from the experiments also showed that the effects were derived from the increased amount of propionic acid in the colon delivered by the cleavage of the propionic esters and not fermentation of the inulin. A control group receiving pure inulin did not experience similar effects but gained weight during the experiment. In this way, it was shown that the inulin polymer was working primarily as a carrying platform for the release of the propionic acid, by cleavage of the esters in the colon, where fermentation of dietary fibers normally take place.

Likewise it is known that cellulose propionate can be applied for the delivery of propionic acid to the colon, without the successive fermentation of the cellulose, which reduces the amount of gasses and discomfort of fermentation [6]. Inulin propionate and cellulose propionate are the closets known techniques to starch propionate.

The patent for propionylated inulin [3] and corresponding research [4][7] shows the significant difference that ingestion of propionylated inulin makes compared to ingestion of pure inulin in relation to weight loss and metabolism. The overweight people ingesting pure inulin over 24 weeks gained weight, while those ingesting propionylated inulin lost or retained their weight.

Inulin is a soluble dietary fiber and not a resistant starch, but the effect of soluble fibers and resistant starches in the production of SCFAs are comparable. Both arrive undigested in the colon and are fermented by bacteria to produce comparable amounts of SCFAs [8].

### DISCLOSURE OF INVENTION

The effect and use of inulin propionate and cellulose and propionate are similar to starch propionate, since they all can be orally ingested with the effect to selectively increase the delivery of propionic acid in the colon.

Starch propionate will upon ingestion not be digested and taken up in the small intestine as normal starch, since the starch is esterified and thereby becomes resistant to degradation in the stomach and small intestine. Like resistant starch it escapes degradation and digestion in the small intestine and ends up undegraded in the colon. By fermentation in the colon propionic acid is released by hydrolysation of the ester bonds and releases propionic acid in the colon parallel to the effect of the ingestion of inulin propionate. The de-esterified starch will afterwards be fermented in the colon and functionally works as a “normal” fermentable dietary fiber, which acts as nutrition to the fermenting bacteria. Starch propionate can, like inulin propionate, thus be applied in connection with weight loss, to prevent weight gain, reduce the food- and calorie intake, reduce the intra-abdominal fat distribution, reduce the amount of intraheptaocellular fat and reduce the desire to high energy foods. The new is to use starch propionate in connection with weight loss, to prevent weight gain, reduce the food- and calorie intake, reduce the intraabdominal fat distribution, reduce the amount of intraheptaocellular fat and reduce the desire to high energy foods. A new product is applied to prevention of overweight and obesity, which is not known within the technical field of delivery of propionic acid to the colon for prevention of overweight and obesity.

By esterification of starch with propionic acid, the starch propionate as mentioned above acquire properties like resistant starch, meaning it arrives undegraded in the colon to be fermented by bacteria. The effect of starch propionate on the level of propionic acid in the colon is however much higher than for “normal” resistant starch. 30 gram of starch propionate (with e.g. 8% propionic acid and a water content below 20%) will selectively increase the amount of propionic acid by 2 gram and additionally also supply 20 gram of starch for possible fermentation. Firstly, esters are cleaved by enzymes found in the colon and subsequently the resulting starch is fermented by the colon bacteria. When fermented in the colon resistant starch produce SCFAs of which approximately 20% is propionic acid. The production ratio of 20% propionic acid out of produced SCFAs from fermentation does not change by increased intake [8]. The daily amount of produced propionic acid by fermentation is estimated to 2.5 grams for a 85 kg healthy person [9]. It has been shown that intake of 28-34 gram (dry weight) of resistant starch purified from potatoes increases the total amount of SCFAs in young healthy individuals by 32% [10]. This corresponds to an increased amount of propionic acid by 0.8 gram. This increased amount of dietary fiber/resistant starch (28-34 gram in dryweight) is at the approximate limit to what is possible to consume of carbohydrates (e.g. dietary fibers/resistant starches) for fermentation in the colon without achieving severe discomfort [10].

Starch propionate consumed in an amount of 30 gram (20% water content, 8% propionic acid) thus increases the total amount of propionic acid in the colon by 2-2.6 gram, where 30 grams of resistant starch (dry weight) only increases the total amount of propionic acid by 0-0.8 gram. Thus, consuming starch propionate is in excess of 3 times as effective in increasing propionic acid present in the colon compared to eating the same amount of resistant starch. The maximum amount of resistant starch or dietary fibers which is possible to consume without getting severe stomach discomfort is 30 grams and only delivers 0.8 grams of propionic acid. This is additionally assuming that the correct composition of bacteria is present in the colon to conduct the fermentation effectively into SCFAs. As discussed above the latter for a variety of reasons is rare in overweight people on a western diet and if the right bacteria composition is not present, the difference in effect will be much higher. If the bacteria can't deliver propionic acid by fermentation propionylated starch will still deliver 2 gram of propionic acid by cleavage of the esters, but not additionally the 0.8 gram, because the resistant starch will not be fermented. Thus, the difference in delivery is 2 grams from starch propionate and 0 grams from resistant starch, so the ratio of efficiency of the delivery of starch propionate in this instance is much larger than 3 times.

Furthermore the last example is the most common for overweight people on western diet. This is because an unfavorable composition of bacteria is likely the cause why the majority of overweight people, who eats an modern western diet, do not loose weight with an increased consumption of resistant starch or soluble fibers as shown in the experiment from Copenhagen University with “New Nordic Diet”.

Thus, the effect of starch propionate can not be compared to the effect of resistant starch and the usage and effects described in claims 1-9 differs from the usage and effects of the consumption of resistant starch. And for this reason, this patent concerns using starch propionate to gain the effects described in claims 1-9. The cleaving of the ester bonds releases free propionic acid in the colon and this mechanism is what primarily increases the amount of propionic acid and makes it possible to use starch propionate as described in claims 1-9. Furthermore the main purpose of the esterification of starch with propionic acid is not to convert starch to “resistant starch” and achieve fermentation in the colon. The main purpose is to achieve liberation of propionic acid directly in the colon in 3 times larger amounts than fermentation allows, independently of the bacterial composition in the colon. The independency is especially important for people on a modern western diet lacking the bacteria for proper fermentation of dietary fibers in the colon.

Inulin propionate and cellulose propionate are the closest known techniques to starch propionate for the delivery of propionic acid to the colon. Even though they are all propionylated carbohydrates, they are dissimilar and far from the same products. They differ in 1. chemistry, 2. solubility, 3. application or usage, 4. price of raw materials and production methods, 5. expected sales price, 6. accessibility for low-income groups due to price, 7. food safety and the possibility of obtaining approval as a novel food. In these 7 ways, starch propionate differs substantially from its closest known alternatives.

1. Chemistry: Inulin consists of joined fructose units and starch of joined glucose units. Cellulose is a linear polymer of glucose units joined by β-1-4 bonds. Starch is a mixture of amylose and amylopectin. The fundamental chemical structure in starch propionate thus differs from inulin propionate and cellulose propionate.

2. Solubility: Inulin is soluble in water, while cellulose and starch is insoluble. This is also true for inulin propionate, celullose propionate and starch propionate, which gives the products different possibilities of application.

3. Application: Propionylated inulin could e.g. be added to soft drinks in soluble form without changing transparency or the appearance of the soft drink. This is not the case for cellulose propionate and starch propionate. Cellulose propionate and starch propionate is insoluble and will cause a change in the appearance of the final product. Propionylated inulin thus has different possibilities of application as a food additive than cellulose propionate and starch propionate. Cellulose is not fermented in the colon and will thus not give gasses and discomfort compared to inulin propionate and starch propionate.

4. Production: Due to propionylated inulin is soluble in water and starch propionate is not, starch propionate can be produced far more simply and efficiently than propionylated inulin. Furthermore starch propionate can be produced on already existing production equipment used in the food additive industry for the production of starch acetylate (E1420). The process is quite similar and comparable, in that propionic anhydride is applied instead of acetic anhydride in an otherwise identical production process on existing production equipment. Reversely for propionylated inulin, the process is complicated and new and no standard equipment exists nor has the exact production line design been conceived yet. Cellulose propionate do not yet have any production process developed for production of it to be used in the food industry.

5. Price: The price for starch propionate will be comparable to production price for starch acetylate at estimated to 5-10 Dkk/kg.(0.75-1.5 $/kg) By ingestion of 30 gram per day of starch propionate, the yearly expense for the consumer will be in the range of 2-400 Dkk (30-60$), when a markup for marketing, retail and profit is added. The biggest material expense for starch propionate is starch, which is extracted very cheaply from potatoes or other sources of starch. This is not the case for propionylated inulin, where costly production facilities are needed and the higher price for inulin will affect the production price. In the patent the production process [3] for inulin propionate is described as follows: Propionic anhydride is reacted with inulin dissolved in water. To separate the by-products (including acid), the reaction mixture is passed through an active coal column, by which the propionylated inulin is collected on the column. Afterwards propionylated inulin is eluted from the column and has to be spray or freeze dried from the soluble form to achieve a powder. Standard price for inulin on sale is between 300-500 dkk/kg (45-75 $/kg). Inulin is extracted from chicory root which is washed, finely grinded, treated with acid and washed again, for the inulin to be washed out. The solution is then carbonized, purified, evaporated into syrup or slurry and finally freeze or spray dried. Because propionylated inulin has additionally process steps on tailor made production equipment, it is estimated that the finished product would have a production price of 500-800 dkk/kg (75-120 $1 kg). The sales price will likely be above 1,000 dkk/kg. (150 $/kg). The estimated consumption is 10 gram per day, and will result in a yearly expense for the consumer in the range of 6,000-10,000 dkk, (900-1,500 $/kg) when a markup for marketing, retail and profit is added. Cellulose is likewise are more expensive starting material than starch with more complicated extraction and purifications steps and is variable in many different qualities. Food quality in whole sales of microcrystal cellulose is approximately 20-35 dkk/kg, which is over ten times the price for whole sales of starch. Is this factor applied in the calculation of a potential yearly expense of cellulose propionate compared to starch propionate, a yearly expense of cellulose propionate would be 2000-5000 dkk.

6. Accessibility for low-income groups: Starch propionate will due to the lower price be accessible for low income individuals—including low-income groups in the western economies and the broad population in the developing countries suffering from obesity and overweight. This could e.g. be middle-income groups in the new emerging economies (Africa, India, South east Asia, central Asia, China, Latin America, Middle East, etc.) where the obesity epidemic is especially widespread. WHO estimated in 2016 that the overweight and obese share of the world population above the age of 18 was approximately 1.9 billion people. Thus starch propionate will due to its low price potentially be accessible for over 2 billion people suffering from overweight or obesity. The higher price for propionylated inulin and cellulose propionate means that they will be accessible only for high-income groups in the emerging economies as well as high- and middle-income groups in the western world. Propionylated inulin and cellulose propionate will however remain significantly cheaper than known drugs for the treatment of overweight and obesity and therefor be economically attractive to market in the western world, as a cost-efficient alternative to existing therapies. It will however not be an economic viable choice for low income individuals in the western world as well as middle income individuals in the emerging economies. Due to its low price starch propionate will fill this gap and will be an economic viable alternative to existing treatments and thus also available for these low-income groups.

7. Food safety and approval: With regards to food safety and approval as a novel food or food additive starch propionate has the advantage that modified starches have been accessible and used in the food industry since the 1960's. In this way, the food safety of comparable products is known and an application for approval as novel food of starch propionate can build on this. As mentioned, starch propionate has the same production process as starch acetylate (E1420). This also is an is an advantage in the approval process of starch propionate because the process needs to be established and well described for this approval. In contrast propionylated inulin, has proven to be difficult to get approved as a novel food in spite of both inulin and propionic acid already approved. The approval of propionylated inulin as a novel food has been submitted but delayed due to food authorities need for further investigations as well as better explanation and justification of the production process. Cellulose propionate due not yet have a developed process for production to be used in the food industry, but properly would involve the use of organic solvents.

The seven points which differentiates starch propionate from inulin propionate, shows conclusively that they are different substances and different solutions to the same problem in certain respects. The price of raw materials, production advantages and the familiarity to known additives and food supplements that starch propionate has allows it to solve a further problem or address a different issue; the accessibility to low income individuals suffering from obesity and overweight. In the end starch propionate solves a problem of general health economic interest. Health economists deals on a daily basis with the issue: “How can you treat millions of overweight and obese people in the world with low yearly incomes, so that the world wide obesity epidemic is dampened and the included catastrophic effects on public health and state budgets?”. This problem starch propionate can solve, but inulin propionate and cellulose propionate can not due to the costs.

### BRIEF DESCRIPTION OF DRAWINGS

No drawings are included in the application.

### BEST MODE FOR CARRYING OUT THE INVENTION

The simplest and best method of use for humans is to drink 10 gram of starch propionate in a slurry of cold water after breakfast, lunch and dinner, so a total amount of 30 gram is consumed daily (0.35 grams per kg bodyweight). Starch propionate in this daily dosage should contain 8% propionic acid attached as esters and a water content below 20%.

The food supplement (starch propionate) can further be added to food such as smoothies, half and whole fabricated dishes designed for dieting and weight control.

### INDUSTRIAL APPLICABILITY

Starch propionate can be used in the food_industry and thus fulfills requirements for industrial applicability where this requirement is necessary for patentability.

Mentioned Publications
1. De Filippo C, Cavalieri D, Di Paola M, et al. Impact of diet in shaping gut microbiota revealed by a comparative study in children from Europe and rural Africa. Proc Natl Acad Sci USA. 2010; 107(33):14691-14696. doi:10.1073/pnas.1005963107
2. Hjorth M F, Roager H M, Larsen T M, et al. Pre-treatment microbial Prevotella-to-Bacteroides ratio, determines body fat loss success during a 6-month randomized controlled diet intervention [published correction appears in Int J Obes (Lond). 2018 Feb. 6;:]. Int J Obes (Lond). 2018; 42(3):580-583. doi:10.1038/ijo.2017.220
3. Patent 2013-07-31PCT/GB2013/052056
4. Chambers E S, Viardot A, Psichas A, et al. Effects of targeted delivery of propionate to the human colon on appetite regulation, body weight maintenance and adiposity in overweight adults. Gut. 2015; 64(11):1744-1754. doi:10.1136/gutjnl-2014-307913.
5. Byrne C S, Chambers E S, Alhabeeb H, et al. Increased colonic propionate reduces anticipatory reward responses in the human striatum to high-energy foods. Am J Clin Nutr. 2016; 104(1):5â€″14. doi:10.3945/ajcn.115.126706
6. DK Patent PA 2020 00170
7. Byrne C S, Chambers E S, Preston T, et al. Effects of Inulin Propionate Ester Incorporated into Palatable Food Products on Appetite and Resting Energy Expenditure: A Randomised Crossover Study. Nutrients. 2019; 11(4):861. Published 2019 Apr. 16. doi:10.3390/nu11040861
8. Cummings, John H.; Macfarlane, George T. da; Englyst, Hans N. (2001). “Prebiotic digestion and fermentation”. Am J Clin Nutr. 73 (suppl): 415S-420S. doi:10.1093/ajcn/73.2.415s. PMID
9. Morrison D J, Preston T. Formation of short chain fatty acids by the gut microbiota and their impact on human metabolism. Gut Microbes. 2016; 7(3):189-200. doi:10.1080/19490976.2015.1134082
10. Baxter N T, Schmidt A W, Venkataraman A, Kim K S, Waldron C, Schmidt T M. 2019. Dynamics of human gut microbiota and short-chain fatty acids in response to dietary interventions with three fermentable fibers. mBio 10:e02566-18. https://doi.org/10.1128/mBio.02566-18.

1. Starch propionate used as a food additive and/or food supplement for humans for prevention of overweight and obesity and/or for weight reduction and/or to prevent weight gain and/or to decrease appetite and/or to enhance sensation of fullness and/or to reduce food and calorie intake and/or to reduce the intraabdominal fat and/or for lowering the amount of intrahepatocellular fat and/or for reducing the desire for high energy foods.

2. Starch propionate used as in claim 1 wherein the sensation of fullness increases and appetite decreases.

3. Starch propionate used as in claim 1 wherein food and calorie intake is reduced.

4. Starch propionate used as in claim 1 wherein the intraabdominal fat distribution is reduced and the amount of intrahepatocellular fat is lowered.

5. Starch propionate used as in claim 1 wherein the desire for high energy foods is reduced.

6. Starch propionate used as in claim 1 wherein it is taken as a suspension or in tablets.

7. Starch propionate used as in claim 1 wherein it is taken in a daily dosage up to 50 grams in a single dosage or in several smaller doses.

8. Starch propionate used as in claim 1 wherein it is used for weight reduction.

9. Starch propionate used as in claim 1 wherein it is used for prevention of weight gain.
